(19) United States
(12) Patent Application Publication
(10) Pub. No.: US 2006/0079720 A1
(43) Pub. Date: Apr. 13, 2006

(54) Method for preparing acetal-containing compositions

(75) Inventors: Chunping Xie, Boiling Springs, SC (US); Jiang Li, Spartanburg, SC (US); Jusong Xia, Moore, SC (US)

Correspondence Address: John E. Vick, Jr.;Legal Department, M-495, PO Box 1926, Spartanburg, SC, 29304, US

(73) Assignee: Milliken & Company

(21) Appl. No.: 10/964,084

(22) Filed: Oct. 13, 2004

(51) Int. Cl. C07C 43/32; C07C 43/30

(52) U.S. Cl. 568/592

(57) ABSTRACT

An acetal compound may be formed by the method of reacting a substitiuted or unsubstituted benzaldehyde, a polyhydric alcohol, and an at least one acid catalyst at ambient temperatures, in a homogenous reaction media in the presence of at least one water miscible organic solvent. The molar ratio of the acid catalyst to the benzaldehyde may be less than about 0.6 to 1, respectively, of acid catalyst to benzaldehyde.

### BACKGROUND OF THE INVENTION

Acetal derivatives of polyhydric alcohols are useful in several applications, including for example as nucleating agents for polymer resins, and as gelling and thickening agents for organic liquids.

The use of nucleating agents to reduce the haze in articles manufactured from crystalline polyolefin resins is known in the art. Representative acetals of sorbitol and xylitol, which have been employed as clarifying agents, are described in several patents, including for example: Hamada, et al., U.S. Pat. No. 4,016,118, dibenzylidene sorbitols; Kawai, et al., U.S. Pat. No. 4,314,039, di(alkylbenzylidene) sorbitols; Mahaffey, Jr., U.S. Pat. No. 4,371,645, diacetals of sorbitol having at least one chlorine or bromine substituent; Kobayashi, et al., U.S. Pat. No. 4,954,291, distribution of diacetals of sorbitol and xylitol made from a mixture of dimethyl or trimethyl substituted benzaldehyde and unsubstituted benzaldehyde. Another reference, U.S. Pat. No. 5,049,605 to Rekers et al. discloses bis(3,4-dialkylbenzylidene) sorbitols, including substituents forming a carbocyclic ring. Dibenzylidene sorbitol (DBS) and substituted DBS are used commercially as nucleating agents in thermoplastics and gelling agents for organic liquids.

Several synthetic methods of DBS compounds have been disclosed in literature. European Patent application 0497976B1 by New Japan Chemical discloses a method to produce dibenzylidene sorbitol (DBS) by condensing an aromatic aldehyde with sorbitol in the presence of a acid catalyst, cyclohexane and methanol under elevated temperature.

Several United States patents have been published pertaining to the manufacture of DBS type compounds. These include U.S. Pat. No. 5,731,474 to Scrivens et al. which is directed to a method of making acetals.

U.S. Pat. No. 6,500,964 to Lever et al. discloses a process utilizing mineral acids and surfactants. This process produces DBS at about 70% yield with purity of 98%, wherein a relatively large amount of acid catalyst is used to produce DBS.

U.S. Pat. No. 5,106,999 to Gardlik (the “Gardlik patent”) discloses a process for preparing DBS compounds. In particular, it discloses a process for preparing meta-substituted halogenated derivatives by reacting D-sorbitol with benzaldehydes. In this process, methanol and a protonic acid are used. The ratio of acid catalyst to aromatic aldehyde disclosed in the patent is from 0.6:1 to about 1.5:1, and preferably about 0.7:1.

There are disadvantages of the methods to synthesize DBS compounds taught by the prior art. In the process involving cyclohexane-methanol as the reaction media, heating is required due to the relative low efficiency of the reaction caused by the two-phase solvent system. In the process using water as the medium, surfactant is required to make the phase transfer possible, which in turn makes the reaction occur. The presence of surfactants makes the purification more complicated. In the process for DBS manufacture disclosed in the Gardlik patent and Level patent, the use of relatively large amounts of acid catalyst may be necessary, resulting in a more difficult purification procedure, equipment damage and higher cost. Using these methods, at the conclusion of the reaction, it is typically required that the acid be removed, and the DBS product purified. Therefore, the large amount of acid required in this process makes the purification of the final DBS product more difficult and more expensive. In general, the more acid used, the more undesirable and inefficient the process.

What is needed in the chemical industry is a better, more efficient method for the manufacture of acetals of polyhydric alcohol type compounds. A method that avoids the use of complicated solvent systems, large amounts of energy, and large amounts of acid catalysts while still achieving very high efficiency would be desirable. Furthermore, it would be helpful to expand the scope of DBS synthesis by using other types of acids that are not protonic-type acids as the catalysts. The invention is directed to solving some of these problems in the industry, and is further described herein.

### DETAILED DESCRIPTION OF THE INVENTION

In the invention, a novel, efficient and convenient method is provided for the synthesis of acetals of polyhydric alcohols. This process may be used for allyl, alkyl, halogen, or other substituted or unsubstituted derivatives of DBS.

An acetal compound may be formed in one particular embodiment of the invention by the process of condensation of at least one polyhydric alcohol with at least one aromatic aldehyde, in the presence of at least one acid catalyst at a low level, to form at least one acetal compound. However, the invention may be practiced in other ways as well. The acetal compound formed may be a mono-, di-, or tri-acetal, but in many cases it has been found that a di-acetal is particularly useful.

In this invention, a method of forming an acetal of a polyhydric alcohol is shown by reacting in a homogeneous reaction media:
(a) a substituted or unsubstituted benzaldehyde;
(b) a polyhydric alcohol;
(c) at least one water-miscible organic solvent; and
(d) at least one acid catalyst.

In some embodiments of the invention, there may be provided an initial reaction molar ratio of acid catalyst to benzaldehyde of less than about 0.6:1, respectively. A useful initial molar ratio of acid catalyst to aromatic aldehyde is 0.3:1, or less. In some applications, the molar ratio of acid catalyst to aromatic aldehyde may be 0.15:1, or less.

The acid catalyst might be a protonic acid or a Lewis acid, or the mixture thereof. The protonic acid may be selected from the group consisting of hydrochloric acid, sulfuric acid, phosphoric acid, and mixture thereof.

The Lewis acid may be selected from among essentially any acid capable of receiving electrons, including, for example a bismuth-containing compound. For purposes of this disclosure, a Lewis acid is any species with a vacant orbital, which can accept a pair of electrons. Lewis acids are believed to be especially useful in the practice of the invention. Examples of Lewis Acids that can be used are provided below: AlCl₃, ZnCl₂, SnCl₂, SnCl₄, SnBr₂, SnBr₄, Bi(OTf)₃, MgBr₂, FeCl₃, BF₃.

The organic solvents suitable for the inventive process are preferably water miscible, such as C1-C10 alcohols, acetonitrile, tetrahudrofuran, dioxane, and mixtures thereof.

This invention relates to a process for preparing alditol acetals, such as dibenzylident sorbitols, monobenzylidene sorbitols and the like, through the reaction of unsubstituted or substituted aromatic aldehydes with alditols (such as xylitol, sorbitol, substituted xylitol, such as alkyl xylitol, alkenyl xylitol, or substituted sorbitol, such as alkyl sorbitol, alkenyl sorbitol) in the presence of at least one water-miscible organic solvent (such as acetonitrile, 1,4-dioxane, nitromethane and methanol), and an acid catalyst, at room temperature.

For purposes of this specification, “water-miscible organic solvent” refers to an organic solvent that forms a one-phase system when mixing with water at any ratios. With small amounts of acid catalyst usage, this procedure provides a mild, cost-effective, highly efficient approach in a homogeneous reaction media with easy purification. “Homogeneous reaction media” refers to a one-phase solvent system that is composed of one or more solvents that are miscible.

Such a reaction is able to synthesize some diacetals (such as diacetals from ortho halogen-substituted benzaldehydes), which are not accessible by other methods (for example: cyclohexane-methanol shots reaction). Such alditol acetals are useful as nucleating and clarifying agents for polyolefin formulations and gellator for cosmetic industry.
Reaction Scheme:

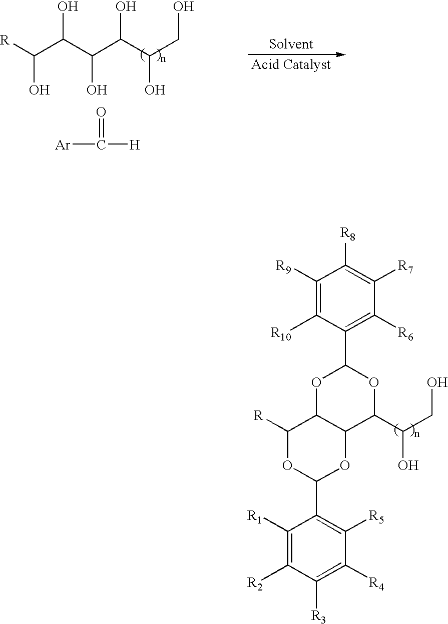

For the above scheme, a homogenous reaction media containing at least one organic solvent is employed. The reaction media includes a water-miscible organic solvent (such as acetonitrile, 1,4-dioxane, nitromethane, ethanol, and methanol, as examples) or mixtures thereof, with or without water.

The acid catalyst may be protonic acid (such as p-toluenesulfonic acid (pTSA), or hydrochloric acid) or one of many different types of Lewis acids, such as bismuth triflate, tin(II) bromide, tin(IV) bromide), or mixtures thereof. In general, n is 0, 1, or 2.

R is independently selected from hydrogen, alkenyl (such as allyl), alkyl, alkoxy, hydroxylalkyl, alkyl-halide, aromatic and substituted aromatic groups.

R₁, R₂, R₃, R₄, R₅, R₆, R₇, R₈, R₉, and R₁₀ are independently selected from the group consisting of hydrogen, fluorocarbons, alkenyl, alkyl, alkynyl, alkoxy, carboxy, halides, amino, thioether and aromatic groups, or in some embodiments of the invention, any two adjacent groups may be combined to form a cyclic group, wherein said cyclic group may be comprised of methylenedioxy, cyclopentyl, cyclohexyl, or other similar cyclic groups.

In one practice of the invention, a process is provided for reacting (a) and (b) below

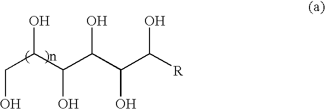

wherein R is independently selected from non-hydrogen groups including alkenyl (including allyl), alkyl, alkoxy, hydroxyl alkyl, and alkyl-halide, aromatic groups; and

wherein n comprises 0, 1, or 2; and 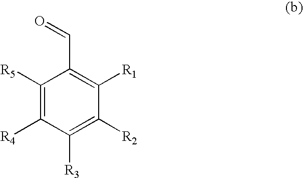
wherein R₁, R₂, R₃, R₄, and R₅ are independently selected from the group consisting of hydrogen, fluorocarbons, alkenyl, alkyl, alkynyl, alkoxy, carboxy, halides, amino, thio ether and aromatic groups; in a homogenous reaction media that contains:

(c) at least one water-miscible organic solvent; and

(d) at least one protonic acid, or Lewis acid catalyst, or mixture thereof;

wherein the initial molar ratio of acid catalyst to aromatic aldehyde is less than 0.6:1.

A compound may be formed as such:

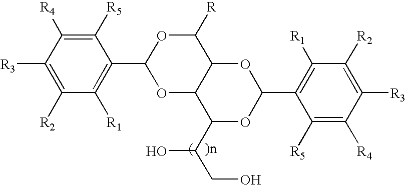

In another method, an unsubstitited or substituted DBS may be formed by reacting in a homogenous reaction media, a substituted or unsubstituted benzaldehyde; a polyhydric alcohol; at least one water-miscible organic solvent; and a Lewis acid; wherein the reaction forms DBS. The reaction may occur at ambient temperatures, in most cases, depending upon the particular Lewis acid chosen.

In some applications, such a reaction product or resulting composition may be a di-acetal (and thus the result of a 1:2 molar ratio reaction between the alditol and benzaldehyde). A composition may be provided having the structure of Formula (III), below. A mono acetal, or a triacetal, could also be provided in the practice of the invention. The di-acetal composition is shown below:

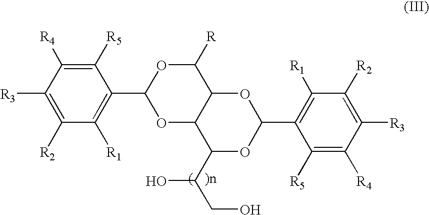

It should be appreciated that the R group stereochemistry is not defined, and the invention is not limited to any particular R group stereochemistry, such that all chemical structures provided herein shall cover any isomers that occur due to stereoisomers of the carbon atom to which R is attached.

It should be appreciated with regard to the composition set forth above that while only the 1,3; 2:4 isomer is represented (i.e. the numbered carbons on the sorbitol chain which form the two acetals), this structure is provided for convenience and illustration only and the invention is not limited to only isomers of the 1,3:2,4 type, but may include any and all other isomers as well, including also isomers of the 1:3; 4:6 and 2:4; 3:5 type, as examples.

The diacetals, triacetals, and monoacetals of the invention may be condensation products of unsubstituted alditols, such as (but not limited to) sorbitol and xylitol, or substituted alditols, such as (but not limited to) allyl-sorbitol, propyl-sorbitol, 1-methyl-2-propenyl sorbitol, allyl-xylitol, propyl-xylitol, and a (substituted) benzaldehyde. Examples of suitable (substituted) benzaldehydes include benzaldehyde, 4-ethylbenzaldehyde, 4-isobutylbenzaldehyde, 4-fluoro-3-methylbenzaldehyde, 5,6,7,8-tetrahydro-2-naphthaldehydebenzylidene, 3-methylbenzaldehyde, 4-propylbenzaldehyde, 4-butylbenzaldehyde, 4-methoxybenzaldehyde, 3-chlorobenzaldehyde, 3,4-dimethylbenzaldehyde, 3,5-difluorobenzaldehyde, 3-fluorobenzaldehyde, 4-fluorobenzaldehyde, 3-bromo-4-fluorobenzaldehyde, 3-methyl-4-methoxybenzaldehyde, 2,4,5-trimethylbenzaldehyde, 4-chloro-3-fluorobenzaldehyde, 4-methylbenzaldehyde, 3-bromobenzaldehyde, 4-methoxybenzaldehyde, 3,4-dichlorobenzaldehyde, 4-fluoro-3,5-dimethylbenzaldehyde, 2,4-dimethylbenzaldehyde, 4-bromobenzaldehyde, 3-ethoxybenzaldehyde, 4-allyloxybenzaldehyde, 3,5-dimethylbenzaldehyde, 4-chlorobenzaldehyde, 3-methoxybenzaldehyde, 4-(trifluoromethyl)benzaldehyde, 2-naphthaldehyde, 4-isopropylbenzaldehyde, 3,4-diethoxybenzaldehyde, 3-bromo-4-ethoxybenzaldehyde, piperonal, 3,4-dimethoxybenzaldehyde, 4-carboxybenzaldehyde, 3-hex-1-ynylbenzaldehyde, and 2-chlorobenzaldehyde. Preferred di-acetals of the present invention include 1,3:2,4-bis(3′,4′-dimethylbenzylidene) sorbitol, 1,3:2,4-bis(benzylidene) sorbitol, 1,3:2,4-bis(4′-methylbenzylidene) sorbital, 1,3:2,4-bis(4-ethylbenzylidene)-1-allyl-sorbitol, 1,3,2,4-bis(3′-methyl-4′-fluoro-benzylidene)-1-propyl-sorbitol, 1,3,2,4-bis(5′,6′,7′,8′-tetrahydro-2-naphthaldehydebenzylidene)-1-allyl-xylitol, bis-1,3,2-4-(3′,4′-dimethylbenzylidene)-1″-methyl-2″-propyl-sorbitol, 1,3,2,4-bis(3′,4′-dimethylbenzylidene)-1-propyl-xylitol, as examples.

The following examples are illustrative of the invention, but do not limit the scope of the invention. Species provided below may enable a person of skill in the art to practice the entire chemical genus represented by the specific species presented below.

### EXAMPLE 1

### 1,3:2,4-Bis(3′,4′-dimethylbenzylidene) Sorbitol

To the white slurry of D-sorbitol (9.11 g, 50 mmol) and 3,4-dimethylbenzaldehyde (13.4 g, 100 mmol) in acetonitrile (100 mL) at room temperature was added a solid of p-toluenesulfonic acid monohydrate (1.9 g, 10 mmol). After magnetically stirring for 12 h, the gel-like material (no visible solvent present) was washed sequentially with boiling water (200 mL×2), cyclohexane (200 mL×2) and boiling water (200 mL×4). After drying in vacuum oven at 110° C. for 12 h, 1,3:2,4-bis(3′,4′-dimethylbenzylidene) sorbitol (20.5 g, 99%) was obtained as a white powder. The product was properly characterized using ¹H and ¹³C NMR, IR and GC/MS.

### EXAMPLE 2

### 1,3:2,4-Bis(3′,4′-dimethylbenzylidene) Sorbitol

The target molecule was synthesized using similar procedure as described in Example 1 with D-sorbitol (9.11 g, 50 mmol), 3,4-dimethylbenzaldehyde (13.4 g, 100 mmol), and p-toluensulfonic acid monohydrate (1.9 g, 10 mmol) in 1,4-dioxane (100 mL). After the same purification procedure as described in Example 1, 1,3:2,4-bis(3′,4′-dimethylbenzylidene) sorbitol (11.4 g, 55%) was obtained as a white powder. The product was properly characterized using ¹H and ¹³C NMR, IR and GC/MS.

### EXAMPLE 3

### 1,3:2,4-Bis(3′,4′-dimethylbenzylidene) Sorbitol

The target molecule was synthesized using similar procedure as described in Example 1 with D-sorbitol (9.11 g, 50 mmol), 3,4-dimethylbenzaldehyde (13.4 g, 100 mmol), and p-toluensulfonic acid monohydrate (1.9 g, 10 mmol) in nitromethane (100 mL). After the same purification procedure as described in Example 1, 1,3:2,4-bis(3′,4′-dimethylbenzylidene) sorbitol (11.4 g, 55%) was obtained as a white powder. The product was properly characterized using ¹H and ¹³C NMR, IR and GC/MS.

### EXAMPLE 4

### 1,3:2,4-Bis(3′,4′-dimethylbenzylidene) Sorbitol

The target molecule was synthesized using similar procedure as described in Example 1 with D-sorbitol (9.11 g, 50 mmol), 3,4-dimethylbenzaldehyde (13.4 g, 100 mmol), and p-toluensulfonic acid monohydrate (1.9 g, 10 mmol) in N,N-dimethylformamide (DMF, 100 ml). After the same purification procedure as described in Example 1, 1,3:2,4-bis(3′,4′-dimethylbenzylidene) sorbitol (1.7 g, 8%) was obtained as a white powder. The product was properly characterized using ¹H and ¹³C NMR, IR and GC/MS.

The reaction conditions and the yields of Examples 1-4 are summarized in Table 1.

TABLE 1
Effects of different reaction media

| | | Molar Ratio | Reaction | |
|---|---|---|---|---|
| Example | Catalyst | (Catalyst/benzaldehyde) | Media | Yield |
| 1 | pTSA | 0.1 | Acetonitrile | 99% |
| 2 | pTSA | 0.1 | 1,4-dioxane | 55% |
| 3 | pTSA | 0.1 | Nitromethane | 55% |
| 4 | pTSA | 0.1 | DMF | 8% |

### EXAMPLE 5

### 1,3:2,4-Bis(3′,4′-dimethylbenzylidene) Sorbitol

To the white slurry of D-sorbitol (9.11 g, 50 mmol) and 3,4-dimethylbenzaldehyde (13.4 g, 100 mmol) in methanol (100 mL) at room temperature was added a solid of tin dichloride dihydrate (2.3 g, 10 mmol). After magnetically stirring for 12 h, the gel-like material (no visible solvent present) was washed sequentially with boiling water (200 mL×2), cyclohexane (200 mL×2) and boiling water (200 mL×4). After drying in vacuum oven at 110° C. for 12 h, 1,3:2,4-bis(3′,4′-dimethylbenzylidene) sorbitol (10.3 g, 50%) was obtained as a white powder. The product was properly characterized using ¹H and ¹³C NMR, IR and GC/MS.

### EXAMPLE 6

### 1,3:2,4-Bis(3′,4′-dimethylbenzylidene) Sorbitol

The target molecule was synthesized using similar procedure as described in Example 5 with D-sorbitol (36.4 g, 200 mmol), 3,4-dimethylbenzaldehyde (53.7 g, 400 mmol), and bismuth triflate hydrate (0.1 g, 0.15 mmol) in methanol (400 mL). After the same purification procedure as described in Example 5, 1,3:2,4-bis(3′,4′-dimethylbenzylidene) sorbitol (78.7 g, 95%) was obtained as a white powder. The product was properly characterized using ¹H and ¹³C NMR, IR and GC/MS.

### EXAMPLE 7

### 1,3:2,4-Bis(3′,4′-dimethylbenzylidene) Sorbitol

42.46 grams (0.226 mol) of D-sorbitol, 60.65 grams (0.45 mol, 2 eq) of 3,4-dimethylbenzaldehyde, 47.98 g (0.45 mol, 2 eq) of trimethyl orthoformate and 0.11 g of bismuth triflate hydrate are mixed with 560 ml of dry methanol, and the suspension is heated to reflux for 1 hour to achieve a clear solution. The whole mixture is then stirred at room temperature over the weekend (2 days). The whole flask reaction mixture becomes thick gel-like (solidified), which is then added 300 ml of methanol, and the solid is collected by filtration. After washing 6 times with 6×200 ml of boiling water, the white solid product is dried at room temperature for 2 days, and then dried overnight in a vacuum oven at 110° C. 93 gram (yield 99%) of product is obtained as a white powder, with a GC-MS purity of 99.54% and mp of 260C (dec.).

### EXAMPLE 8

### 1,3:2,4-Bis(3′,4′-dimethylbenzylidene) Sorbitol

42.46 grams (0.226 mol) of D-sorbitol, 60.65 grams (0.45 mol, 2 eq) of 3,4-dimethylbenzaldehyde, 47.98 g (0.45 mol, 2 eq) of trimethyl orthoformate and 0.2 g of bismuth triflate hydrate are mixed with 560 ml of dry methanol, and the suspension is stirred at room temperature for 2 days. The whole flask reaction mixture becomes thick gel-like (solidified). After work up as described above, the product is obtained as white powder at similar yield (99%) with similar purity as described in Example #7.

### EXAMPLE 9

### 1,3:2,4-Bis(3′,4′-dimethylbenzylidene) Sorbitol

The target molecule was synthesized using similar procedure as described in Example 5 with D-sorbitol (9.11 g, 50 mmol), 3,4-dimethylbenzaldehyde (13.4 g, 100 mmol), and p-toluensulfonic acid monohydrate (1.4 g, 7.5 mmol) in methanol (100 mL). After the same purification procedure as described in Example 5, 1,3:2,4-bis(3′,4′-dimethylbenzylidene) sorbitol (19.0 g, 92%) was obtained as a white powder. The product was properly characterized using ¹H and ¹³C NMR, IR and GC/MS.

### EXAMPLE 10

### 1,3:2,4-Bis(3′,4′-dimethylbenzylidene) Sorbitol

The target molecule was synthesized using similar procedure as described in Example 5 with D-sorbitol (9.11 g, 50 mmol), 3,4-dimethylbenzaldehyde (13.4 g, 100 mmol), and concentrated hydrochloric acid (0.5 mL g, 6 mmol) in methanol (100 mL). After the same purification procedure as described in Example 5, 1,3:2,4-bis(3′,4′-dimethylbenzylidene) sorbitol (13.0 g, 63%) was obtained as a white powder. The product was properly characterized using ¹H and ¹³C NMR, IR and GC/MS.

The reaction conditions and the product yields of Examples 5-10 are summarized in Table 2.

TABLE 2
Effects of different acid catalysts

| | | Molar Ratio | |
|---|---|---|---|
| Example | Catalyst | (Catalyst/benzaldehydes) | Yield |
| 5 | SnCl₂ | 0.1 | 50% |
| 6 | Bi(OTf)₃ | 0.0004 | 95% |
| 7 | Bi(OTf)₃ | 0.0004 | 99% |
| 8 | Bi(OTf)₃ | 0.0007 | 99% |
| 9 | pTSA | 0.075 | 92% |
| 10 | HCl | 0.060 | 63% |

### EXAMPLE 11

### 1,3:2,4-Bis(4′-chloro-2′-fluorobenzylidene) Sorbitol

To a white slurry of D-sorbitol (14.4 g, 76.5 mmol) and 4-chloro-2-fluorobenzaldehyde (25.0 g, 153 mmol) in methanol (200 mL) at room temperature was added concentrated HCl aqueous solution (1.2 mL, 14 mmol). After mechanically stirring for 48 h, the viscous white slurry was suction filtered, and the residue was washed sequentially with boiling water (1000 mL×2), cyclohexane (1000 mL×2) and boiling water (1000 mL×4). After drying in vacuum oven at 110° C. for 12 h, 1,3:2,4-bis(4′-chloro-2′-fluorobenzylidene) sorbitol (27.6 g, 78%) was obtained as a white powder. The product was properly characterized using ¹H and ¹³C NMR, IR and GC/MS.

### EXAMPLE 12

### 1,3:2,4-Bis(2′-chlorobenzylidene) Sorbitol

The target molecule was synthesized using similar procedure as described in Example 11 with D-sorbitol (70% aqueous solution, 52.1 g, 200 mmol), 2-chlorobenzaldehyde (56.2 g, 400 mmol), and concentrated hydrochloric acid (3.3 mL, 40 mmol) in methanol (400 mL). After the similar purification procedure as described in Example 11, 1,3:2,4-bis(2′-chlorobenzylidene) sorbitol (50.5 g, 59%) was obtained as a white powder. The product was properly characterized using ¹H and ¹³C NMR, IR and GC/MS.

### EXAMPLE 13

### 1,3:2,4-Bis(2′,3′-dichlorobenzylidene) Sorbitol

The target molecule was synthesized using similar procedure as described in Example 11 with D-sorbitol (70% aqueous solution, 52.1 g, 200 mmol), 2,3-dichlorobenzaldehyde (70.0 g, 400 mmol), and p-toluenesulfonic acid (5.7 g, 30 mmol) in methanol (400 mL). After the similar purification procedure as described in Example 11, 1,3:2,4-bis(2′,3′-dichlorobenzylidene) sorbitol (49.3 g, 50%) was obtained as a white powder. The product was properly characterized using ¹H and ¹³C NMR, IR and GC/MS.

### EXAMPLE 14

### 1,3:2,4-Bis(2′,4′-dichlorobenzylidene) Sorbitol

The target molecule was synthesized using similar procedure as described in Example 11 with D-sorbitol (36.4 g, 200 mmol, 2,4-dichlorobenzaldehyde (70.0 g, 400 mmol), and concentrated hydrochloric acid (16 mL, 200 mmol) in methanol (400 mL). After the similar purification procedure as described in Example 11, 1,3:2,4-bis(2′,4′-dichlorobenzylidene) sorbitol (44.3 g, 45%) was obtained as a white powder. The product was properly characterized using ¹H and ¹³C NMR, IR and GC/MS.

The reaction conditions and yields of Examples 11-14 are summarized in Table 3.

TABLE 3
Summary of bisbenzylidene sorbitol derivatives¹

| | | Molar Ratio | |
|---|---|---|---|
| Example | Benzaldehyde | (Catalyst/benzaldehydes) | Yield |
| 11 | 4-chloro-2- | 0.09 | 78% |
| | fluorobenzaldehyde | | |
| 12 | 2-chlorobenzaldehyde | 0.10 | 59% |
| 13 | 2,3- | 0.075 | 50% |
| | dichlorobenzaldehyde | | |
| 14 | 2,4- | 0.50 | 45% |
| | dichlorobenzaldehyde | | |

¹The attempts to synthesize examples shown in this table using the methods taught by prior art were unsuccessful.

### EXAMPLE 15

### 1,3:2,4-Bis(4′-methylbenzylidene) 1-Allyl Sorbitol

A 3 L, three-necked round bottom flask, equipped with heating mantle, stirrer, nitrogen inlet, and condensor, was charged with 900 mL of ethanol, 150 mL of water, 180 g (1.00 mole) of D-glucose, 119 g (1.00 mole) of tin powder (−100 mesh), and 121 g (1.00 mole) of allyl bromide. The mixture was stirred and slowly heated to reflux—a significant exotherm and gas evolution was observed at 60° C. The gray suspension was stirred at reflux for 16 hours. Heat was removed and the mixture was allowed to cool to room temperature. After filtration, two allyl-sorbitol epimers were detected by GC-MS. Typical ratio for threo-erythro isomers was 6:1. The allyl-sorbitol water-ethanol solution (contained SnBr₂) was used without further purification.

A 2 L reaction kettle, equipped with a stirrer and nitrogen inlet, was charged with the above allyl-sorbitol/SnBr₂ water-ethanol solution. 192 g (1.6 mol) of 4-methylbenzaldehyde was added to the reaction vessel. The clear solution was stirred for 16 hours, during which time a significant amount of white solid formed. The solid was isolated by filtration and boiling with 250 ml of 1M NaOH aqueous solution. The white powder was washed with 7×500 ml of boiling water. The washed powder dried overnight. The powder was then stirred in 500 mL of cyclohexane, heated until boiling, filtered, and washed with 2×250 ml of boiling cyclohexane. The isolated white powder was dried in a vacuum oven to give 72 g of product, m.p. 290-292° C. The purity was above 99%, based on GC-MS. ¹H NMR (300 MHz, DMSO-d₆, ppm): 2.30 (s, 6H), 2.40-2.44 (t, 2H), 3.40-4.08 (m, 7H), 4.38 (t, 1H), 4.80 (d, 1H), 5.11-5.19 (q, 2H), 5.59-5.63 (d, 2H), 5.84-5.89 (m, 1H), 7.16-7.20 (m, 4H), 7.31-7.35 (m, 4H).

### EXAMPLE 16

### Asymmetric benzylidene/2,4-dimethylbenzylidene 1-Allyl Sorbitol

A 3 L, three-necked round bottom flask, equipped with heating mantle, stirrer, nitrogen inlet, and condensor, was charged with 900 mL of ethanol, 150 mL of water, 180 g (1.00 mole) of D-glucose, 119 g (1.00 mole) of tin powder (−100 mesh), and 121 g (1.00 mole) of allyl bromide. The mixture was stirred and slowly heated to reflux—a significant exotherm and gas evolution was observed at 60° C. The gray suspension was stirred at reflux for two days, in which time the reaction mixture turned an orange/brown color. Heat was removed and the mixture was allowed to cool to room temperature. The reaction was neutralized to pH=7 by adding approximately 200 ml of 5M NaOH aqueous solution. The suspension was filtered to remove solids, and the yellow solution was decolorized with multiple treatments of activated carbon. The activated carbon was removed by filtration, and the solvent was removed by rotary evaporation to isolate a white syrup. Typical yield was 200 g with threo-erythro ratio of 6:1, based on GC-MS. The syrup was used without further purification.

A 2 L reaction kettle, equipped with a stirrer and nitrogen inlet, was charged with 111 g (0.50 mol) of 1-allyl sorbitol syrup in 280 ml methanol solution. 9.5 g of pTSA (0.05 mol), 53 g (0.5 mol) of benzaldehyde and 67 g (0.50 mol) of 2,4-dimethylbenzaldehyde were added to the reaction vessel. The clear solution was stirred for 48 hours, during which time a significant amount of white precipitate formed. The powder was isolated by filtration and washed with 250 ml of 1M NaOH aqueous solution. The powder was suspended in water and further neutralized to pH=7 with a small amount of NaOH. The suspension was heated to boiling, then filtered. The white powder was washed with 7×500 ml of boiling water. The washed powder dried overnight. The powder was then stirred in 500 mL of cyclohexane, heated until boiling, filtered, and washed with 2×250 ml of boiling cyclohexane. The isolated white powder was dried in a vacuum oven to give 38.4 g of product, m.p. 234-236° C. Standard analyses of the material indicated that it consisted of a mixture of 1,3-O-(benzylidene):2,4-O-(2,4-dimethylbenzylidene) 1-allyl sorbitol and 1,3-O-(2,4-dimethylbenzylidene):2,4-O-benzylidene 1-allyl sorbitol (85%), 1,3:2,4-bis(benzylidene) 1-allyl sorbitol (5%) and 1,3:2,4-bis(2,4-dimethylbenzylidene) 1-allyl sorbitol (10%).

### EXAMPLE 17

### 1,3:2,4-Bis(3′,4′-Dimethylbenzylidene) 1-Propyl Xylitol

A 5 L three-necked round bottom flask, equipped with heating mantle, stirrer, nitrogen inlet, and condenser, was charged with 1.8 liters of ethanol, 0.3 liters of water, 300 g (2.00 mole) of D-xylose, 242 g (2.04 mole) of tin powder (−325 mesh), and 242 g (2.00 mole) of allyl bromide. The mixture was stirred and slowly heated to reflux—a significant exotherm and gas evolution was observed at 60° C. The gray suspension was stirred at reflux for three days, in which time the reaction mixture turned an orange/brown color. Heat was removed and the mixture was allowed to cool to room temperature. The reaction was neutralized to pH=7 by adding approximately 400 ml of 5M NaOH aqueous solution. The suspension was filtered to remove solids, and the yellow solution was decolorized with multiple treatments of activated carbon. The activated carbon was removed by filtration, and the solvent was removed by rotary evaporation to isolate a white syrup. Typical yield was 320 g. 1H NMR (300 MHz, D₂O, ppm): 2.33-2.39 (m, 2H), 3.55-3.89 (m, 6H), 5.14-5.23 (m, 2H), 5.89 (m, 1H). The syrup was used without further purification.

58 g (0.3 mol) of 1-allyl xylitol syrup was dissolved in 60 ml water. About 0.6 g of platinum (5% weight on activated carbon) was added and the mixture was hydrogenated at room temperature with hydrogen pressure at 60 psi. The reaction was stopped until no hydrogen pressure drop was observed. The solid was filtered. The allyl group of the solution was completely turned into propyl group based on NMR. 10 g (0.6 mol) of 3,4-dimethyl benzaldehyde, 500 ml ethanol, and 50 mL concentrated HCl (12N) were added into the sugar solution. The clear solution was stirred at room temperature overnight, during which time a significant amount of white precipitate formed. The powder was isolated by filtration and washed with 100 ml of 1M NaOH aqueous solution. The powder was suspended in water and further neutralized to pH=7 with a small amount of NaOH. The suspension was heated to boiling, then filtered. The white powder was washed with 7×500 ml of boiling water. The washed powder dried overnight. The powder was then stirred in 500 mL of cyclohexane, heated until boiling, filtered, and washed with 2×250 ml of boiling cyclohexane. The isolated white powder was washed with methanol, dried in a vacuum oven to give 21 g of product, m.p. 255-257° C. The purity was above 98%, based on GC-MS. ¹H NMR (300 MHz, DMSO-d₆, ppm): 0.89-0.93 (t, 3H), 1.30-1.50 (m, 2H), 1.50-1.70 (m, 2H), 2.22 (12H), 3.50-4.05 (m, 6H), 4.78 (1H), 5.56-5.59 (d, 2H), 7.14-7.21 (m, 6H).

Purification

Purification of a di-acetal may be accomplished, in one embodiment of the invention, by removal of any present tri-acetals by the extraction thereof with a relatively non-polar solvent. As one non-limited example, by removal of the impurities, the product may be purified so that the amount of di-acetal in the additive composition contains at least about 95 percent and even up to 98 percent di-acetal or more, depending upon the application.

Nucleating Agents and Their Use in Polymers

Olefin polymers which can be nucleated by such compositions include homopolymers and copolymers of aliphatic mono-olefins containing from 2 to about 6 carbon atoms, which have an average molecular weight of from about 10,000 to about 2,000,000, preferably from about 30,000 to about 300,000, such as polyethylene, including linear low density polyethylene, low density polyethylene and high density polyethylene, polypropylene, crystalline ethylene/propylene copolymer (random or block), poly(1-butene) and polymethylpentene.

Examples of other thermoplastic polymer resins which may be nucleated with the disclosed acetal compounds include polyester, poly(ethylene terephthalate) (PET) and poly(butylene terephthalate) and polyamide, including nylon 6 and nylon 6,6, poly(phenylene sulfide), syndiotactic polystyrene and polyketones having carbonyl groups in their backbone.

The compositions made using the process of the invention may be used in a polymer selected from aliphatic polyolefins and copolymers containing at least one aliphatic olefin and one or more ethylenically unsaturated comonomers and at least one mono-, di-, or tri-acetal of substituted alditol (such as allyl-sorbitol, propyl-sorbitol, allyl-xylitol, propyl-xylitol and the like).

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

1. A method of forming an acetal of a polyhydric alcohol using reduced amounts of acid catalyst, said method comprising the reaction of:
(a) at least one substituted or unsubstituted benzaldehyde;
(b) at least one polyhydric alcohol;
(c) a homogenous reaction media containing at least one water-miscible organic solvent; and
(d) at least one acid catalyst;
wherein the initial molar ratio of said acid catalyst to said benzaldehyde is less than about 0.6:1, respectively.

2. The method of claim 1 wherein said acid catalyst is a Lewis acid.

3. The method of claim 2 wherein said Lewis acid is selected from the group of acids consisting of: AlCl₃, ZnCl₂, SnCl₂, SnCl₄, SnBr₂, SnBr₄, Bi(OTf)₃, MgBr₂, FeCl₃, and BF₃, and mixtures thereof.

4. The method of claim 3 wherein said Lewis acid comprises at least Bi(OTf)₃.

5. The method of claim 1 wherein said acid catalyst comprises a mineral acid.

6. The method of claim 5 wherein said mineral acid is selected from the group consisting of: hydrochloric acid, sulfuric acid, phosphoric acid, and mixtures thereof.

7. The method of claim 1 wherein said acid catalyst comprises at least one organic acid.

8. The method of claim 7 wherein said organic acid is selected from the group consisting of para-toluenesulfonic acid, benzenesulfonic acid, 5-sulfosalicylic acid, and naphthalenesulfonic acid, and mixtures thereof.

9. The method of claim 1 wherein said acid catalyst comprises an organic acid, a mineral acid, a Lewis acid, or mixtures of one or more of said acids.

10. The method of claim 1 wherein said reaction occurs at ambient temperatures.

11. The method of claim 10 wherein said water-miscible organic solvent is selected from the group consisting of: C₁-C₁₀ alcohols, acetonitrile, nitromethane, tetrahydrofuran, dioxane, and mixtures thereof.

12. The method of claim 11 wherein said organic solvent comprises a C₁-C₁₀ alcohol, said alcohol being selected from the group consisting of methanol, ethanol, isopropanol, butanol, and mixtures thereof.

13. The method in claim 1 wherein the molar ratio of acid catalyst to said benzaldehyde is less than about 0.2:1, respectively.

14. The method of claim 1 wherein said organic solvent comprises methanol.

15. The method of claim 14 wherein said acid catalyst comprises a Lewis acid.

16. A method of forming an acetal of a polyhydric alcohol, said method comprising the reaction of:
(a) a substituted or unsubstituted benzaldehyde;
(b) a polyhydric alcohol;
(c) a homogenous reaction media containing at least one water-miscible organic solvent; and
(d) at least one Lewis acid catalyst.

17. The method of claim 16 wherein said the initial molar ratio of said Lewis acid catalyst to said benzaldehyde is less than about 0.6:1, respectively; further wherein said Lewis acid catalyst is selected from the group of acids consisting of: AlCl₃, ZnCl₂, SnCl₂, SnCl₄, SnBr₂, SnBr₄, Bi(OTf)₃, MgBr₂, FeCl₃, and BF₃, and mixtures thereof.

18. The method of claim 17 wherein said Lewis acid comprises at least Bi(OTf)₃.

19. The method of claim 16 wherein said homogenous reaction media further comprises a mineral acid.

20. The method of claim 19 wherein said mineral acid is selected from the group consisting of: hydrochloric acid, sulfuric acid, phosphoric acid, and mixtures thereof.

21. The method of claim 16 wherein said homogenous reaction media further comprises at least one organic acid.

22. The method of claim 21 wherein said organic acid is selected from the group consisting of para-toluenesulfonic acid, benzenesulfonic acid, 5-sulfosalicylic acid, and naphthalenesulfonic acid, and mixtures thereof.

23. The method of claim 16 wherein said reaction occurs at ambient temperatures.
